(19) United States
(12) Patent Application Publication
(10) Pub. No.: US 2022/0281910 A1
(43) Pub. Date: Sep. 8, 2022

(54) DINUCLEOTIDE COMPOUNDS FOR TREATING CANCERS AND MEDICAL USES THEREOF

(71) Applicant: PINOTBIO, INC., Gyeonggi-do (KR)

(72) Inventors: Doo-Young JUNG, Daejeon (KR); Jin-Soo LEE, Gyeonggi-do (KR); Hyun-Yong CHO, Gyeonggi-do (KR)

(21) Appl. No.: 17/630,240

(22) PCT Filed: Jul. 29, 2020
(86) PCT No.: PCT/KR2020/010006

§ 371 Date: Jan. 26, 2022

(30) Foreign Application Priority Data
Jul. 30, 2019 (KR) 10-2019-0092688

(51) Int. Cl. C07H 21/04; A61P 35/00

(52) U.S. Cl. CPC C07H 21/04; A61P 35/00

(57) ABSTRACT

The present disclosure provides the dinucleotide compounds which is useful for treating various cancers. The present disclosure also provides a composition comprising the compound or its pharmaceutically acceptable salt. The present disclosure also provides a medical use of the compound, its salt or the composition comprising the compound or its pharmaceutically acceptable salt for treating cancer. The present disclosure also provides a method of treatment of cancer comprising administering the compound, its salt or the composition comprising the compound or its salt to a subject in need of such treatment.

### TECHNICAL FIELD

The present disclosure relates to a group of compounds having an activity inhibiting various cancers. The present disclosure also relates to pharmaceutical compositions comprising the compound(s). The present disclosure relates to methods useful for treating a cancer or tumor, using the compound(s). That is, the present disclosure relates to medical-uses of those compounds according to the present disclosure for treating a cancer or tumor. The present disclosure also relates to a manufacturing method of some dinucleotide compounds.

### BACKGROUND ART

Gemcitabine is a chemotherapy medication used to treat a number of types of cancer. These cancers include breast cancer, ovarian cancer, non-small cell lung cancer, pancreatic cancer, and bladder cancer. Gemcitabine is in the nucleoside analog family of medication. It works by blocking the creation of new DNA, which results in cell death. It is given by slow injection into a vein due to some side effects. Therefore, there has been a constant demand for an anticancer agent based on nucleoside structures, which is less toxic and more effective, and can be administered by other routes of administration.

### DISCLOSURE OF INVENTION

### Technical Problem

Thus one object of the present disclosure is to provide a compound having better anti-cancer activity and/or (physicochemical or pharmacokinetic) property than known nucleoside analogs, pharmaceutical compositions comprising the compound as an active ingredient (effective agent), and medical-uses thereof for treating or preventing cancers.

Another object of the present disclosure is to provide a method for treating or ameliorating cancer comprising administering to a subject in need of treatment, amelioration or prevention of cancer the compound according to the present disclosure.

Yet another object of the present disclosure is to provide a manufacturing method of the compounds according to the present invention.

### SUMMARY

### Solution to Problem

To achieve the object, in one embodiment, there is provided a compound of Chemical Formula 1 or 2:

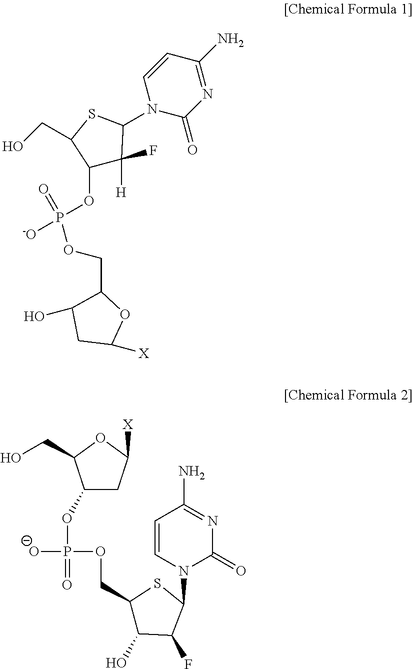

or a pharmaceutically acceptable salt thereof,

in Chemical Formulae 1 and 2, X is adenine, guanine, cytosine or thymine.

In another embodiment, there is provided a pharmaceutical composition comprising a compound of Chemical Formula 1 or 2, or a pharmaceutically acceptable salt thereof, and a pharmaceutically-acceptable carrier or additive.

In yet another embodiment, there is provided a method for treating a cancer comprising administering to a subject a therapeutically effective amount of a compound of Chemical Formula 1 or 2, or a pharmaceutically acceptable salt thereof. The cancer includes, but is not limited to, brain tumor, non-small cell lung cancer, acute myelogenous leukemia, stomach cancer, kidney cancer, colon cancer, prostate cancer, ovarian cancer, skin cancer, or sarcoma. The compound of Chemical Formula 1 or 2, or a pharmaceutically acceptable salt thereof according to the present disclosure is also useful in preventing metastasis and recurrence of tumor by targeting cancer stem cells. That is, there is provided medical-uses of the compound of Chemical Formula 1 or 2, or a pharmaceutically acceptable salt thereof for treating the cancer like what mentioned above.

In yet another embodiment, there is provided a manufacturing method of the compound of Chemical Formula 1 or a pharmaceutically acceptable salt thereof.

The compounds, the pharmaceutical composition, and their medical use above are more fully described in the detailed description that follows.

### DETAILED DESCRIPTION

### The following description is merely exemplary in nature and is not intended to limit the present disclosure, application, or uses.

Definitions

The generic terms used in the present disclosure are herein defined for clarity.

This specification uses the terms “substituent”, “radical”, “group”, “moiety”, and “fragment” interchangeably.

As used herein, the term “patient” means an animal, preferably a mammal such as a non-primate (e.g., cow, horse, sheep, pig, chicken, turkey, quail, cat, dog, mouse, rat, rabbit or guinea pig) or a primate (e.g. , monkey and human), most preferably a human.

As used herein, the term “pharmaceutically acceptable salt(s)” refers to a salt prepared from active compounds according to the present disclosure with relatively non-toxic bases. Base-added salts can be obtained by contacting the neutral compounds with a sufficient amount of the desired base and a pure or inert solvent. Suitable pharmaceutically acceptable base addition salts include, but are not limited to sodium, sodium hydroxide, potassium, potassium hydroxide, calcium, calcium hydroxide, aluminum, organic amino, magnesium, magnesium hydroxide, zinc hydroxide, ammonia, arginine, benethamine, benzathine, choline, deanol, diethylamine, ethanolamine, ethylenediamine, glucamine, hydrabamine, imidazole, lysine, morpholine, piperazine, pyrrolidine, secondary maines, trimethylamine, tromethamine salts and the like.

As used herein, the term “effective amount” includes that amount of a compound of this disclosure sufficient to destroy, modify, control or remove a primary, regional or metastatic cancer cell or tissue; delay or minimize the spread of cancer; or provide a therapeutic benefit in the treatment or management of cancer, a neoplastic disorder, or tumor. An “effective amount” also includes the amount of a compound of this disclosure sufficient to result in cancer or neoplastic cell death.

As used herein, the term “prophylactically effective amount” refers to the amount of a compound sufficient to prevent the recurrence or spread of cancer or the occurrence of cancer in a patient, including but not limited to those predisposed to cancer or previously exposed to a carcinogen.

As used herein, the term “neoplastic” means an abnormal growth of a cell or tissue (e.g., a tumor) which may be benign or cancerous.

As used herein, the term “prevention” includes the prevention of the recurrence, spread or onset of cancer in a patient.

As used herein, the term “treatment” includes the eradication, removal, modification, or control of primary, regional, or metastatic cancer tissue; and the minimizing or delay of the spread of cancer.

As used herein, the phrase “Compound(s) of this/the Disclosure” includes any compound(s) of Chemical Formula 1 and 2, as well as clathrates, hydrates, solvates, or polymorphs thereof. And, even if the term “Compound(s) of the Disclosure” does not mention its pharmaceutically acceptable sat, the term includes salts thereof. In one embodiment, the compounds of this disclosure include stereo-chemically pure compounds, e.g., those substantially free (e.g., greater than 85% ee, greater than 90% ee, greater than 95% ee, greater than 97% ee, or greater than 99% ee) of other stereoisomers.

As used herein, the term “polymorph” refers to solid crystalline forms of a compound of this disclosure or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and/or acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.

As used herein, the term “purified” means that when isolated, the isolate is greater than 90% pure, in one embodiment greater than 95% pure, in another embodiment greater than 99% pure and in another embodiment greater than 99.9% pure.

The term “pharmaceutically-acceptable” means suitable for use in pharmaceutical preparations, generally considered as safe for such use, officially approved by a regulatory agency of a national or state government for such use, or being listed in South Korea or the U. S. Pharmacopoeia or other generally recognized pharmacopoeia for use in animals, and more particularly in humans.

Compounds of the Present Disclosure

There is provided a compound of Chemical Formula 1 or 2:

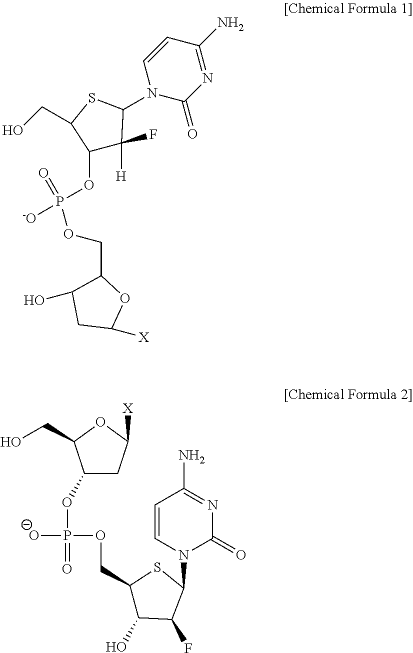

or a pharmaceutically acceptable salt thereof,

in Chemical Formula 1 or 2, X is adenine, guanine, cytosine or thymine.

That is, in Chemical Formula 1 or 2, X is any one of the followings:

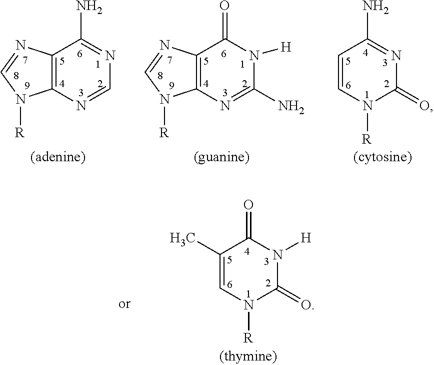

Preferably, there is provided a compound of Chemical Formula 1 above, wherein X is guanine.

The inventors have found out that there are several things to improve in using known nucleoside analogs as an active ingredient of anti-cancer drug. For example, too fast metabolism and too high toxicity need to be improved. In addition, the pharmacokinetic property of known nucleoside analogs is not preferable. The dinucleotide of the present disclosure have much better properties in several aspects for being used as an active ingredient. Particularly, some nucleoside analogs are a general cytotoxic drug and is known to act as a monomer. Thus, it is surprising that the dinucleotides of the present disclosure have such superior activities and properties.

In yet another embodiment, there is provided a pharmaceutical composition comprising a therapeutically effective amount of a compound of Chemical Formula 1 or 2, or a pharmaceutically acceptable salt thereof, and a pharmaceutically acceptable carrier.

In another embodiment, there is provided a method for treating a cancer comprising administering to a subject in need thereof a therapeutically effective amount of a compound of Chemical Formula 1 or 2, or pharmaceutically acceptable salt thereof. The cancer includes, but is not limited to, brain tumor, non-small cell lung cancer, acute myelogenous leukemia, stomach cancer, kidney cancer, colon cancer, prostate cancer, ovarian cancer, skin cancer, or sarcoma. Preferably, the cancer is skin cancer, prostate cancer, non-small cell lung cancer, or acute myelogenous leukemia. In another embodiment, the subject is a human.

That is, there is provided a medical use of Chemical Formula 1 or 2, or pharmaceutically acceptable salt thereof, wherein Chemical Formula 1 or 2, or pharmaceutically acceptable salt thereof is used as an effective agent. In one embodiment, the medical-use is for treatment or prevention of the cancer descried above.

Medical uses and Methods of Treatment of the Compounds according to the Present Disclosure

The present disclosure further provides methods for treating a disease or condition in a subject having or susceptible to having such a disease or condition, by administering to the subject a therapeutically-effective amount of one or more compounds as described above. In one embodiment, the treatment is preventative treatment. In another embodiment, the treatment is palliative treatment. In another embodiment, the treatment is restorative treatment.

1. Diseases or Conditions

The compound of the present disclosure can be used for treating a tumor or cancer, or for preventing aggravation of such disease. Thus, the present disclosure provides a method for inhibiting or hindering cancer cells, wherein the cells are contacted with an effective amount of a compound of the present disclosure. In one embodiment, such cell is present in a subject (for example, cancer patients). In another embodiment, there is provided a medical use for treating a cancer or preventing proliferation of tumor in a subject, using the compound according to the present disclosure. The method of the present disclosure comprises administering to a subject in need of treatment or prevention a pharmaceutical composition containing a therapeutically or prophylactically effective amount of the dinucleotide compound according to the present disclosure.

In one embodiment, there is provided a method for inhibiting a tumor or cancer cell. For example, the present disclosure is used for inhibiting the tumor or cancer cell such as brain tumor cell, non-small cell lung cancer cell, acute myelogenous leukemia cell, stomach cancer cell, kidney cancer cell, colon cancer cell, prostate cancer cell, ovarian cancer cell, skin cancer cell, or sarcoma cell. In this method, the present disclosure provides a method for inhibiting the growth or proliferation of cells, particularly tumor or cancer cells, in a subject. In this method, tumor cells are present in vivo. The compound of the present disclosure can be administered to the subject as a form of the pharmaceutical composition described herein.

In another embodiment, there is provided a method for treating or preventing a cancer or tumor in a subject. The cancer includes, but is not limited to, brain tumor, non-small cell lung cancer, acute myelogenous leukemia, stomach cancer, kidney cancer, colon cancer, prostate cancer, ovarian cancer, skin cancer, or sarcoma. The method comprises administering to a subject in need of treatment an enough amount of the compound, that is, a therapeutically amount of the compound of the present disclosure.

2. Subjects

Suitable subjects to be treated according to the present disclosure include mammalian subjects. Mammals according to the present disclosure include, but are not limited to, human, canine, feline, bovine, caprine, equine, ovine, porcine, rodents, lagomorphs, primates, and the like, and encompass mammals in utero. Subjects may be of either gender and at any stage of development.

In one embodiment, the suitable subject to be treated according to the present disclosure is human.

3. Administration and dosing

The compounds of the present disclosure are generally administered in a therapeutically effective amount.

The compounds of the present disclosure can be administered by any suitable route in the form of a pharmaceutical composition adapted to such a route, and in a dose effective for the treatment intended. An effective dosage is typically in the range of about 0.001 to about 100 mg per kg body weight per day, preferably about 0.01 to about 50 mg/kg/day, in single or divided doses. Depending on age, species and disease or condition being treated, dosage levels below the lower limit of this range may be suitable. In other cases, still larger doses may be used without harmful side effects. Larger doses may also be divided into several smaller doses, for administration throughout the day. Methods for determining suitable doses are well known in the art to which the present disclosure pertains.

Pharmaceutical Compositions, Dosage Forms and Administration Routes

For the treatment of the diseases or conditions referred to the above, the compounds described herein or pharmaceutically acceptable salts thereof can be administered as follows:

Oral Administration

The compounds of the present disclosure may be administered orally, including by swallowing, so that the compound enters the gastrointestinal tract, or absorbed into the blood stream directly from the mouth (e.g., buccal or sublingual administration).

Suitable compositions for oral administration include solid, liquid, gel or powder formulations, and have a dosage form such as tablet, lozenge, capsule, granule or powder.

Compositions for oral administration may be formulated as immediate or modified release, including delayed or sustained release, optionally with enteric coating.

Liquid formulations can include solutions, syrups and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

In a tablet dosage form the amount of drug present may be from about 0.05% to about 95% by weight, more typically from about 2% to about 50% by weight of the dosage form. In addition, tablets may contain a disintegrant, comprising from about 0.5% to about 35% by weight, more typically from about 2% to about 25% of the dosage form. Examples of disintegrants include, but are not limited to, lactose, starch, sodium starch glycolate, crospovidone, croscarmellose sodium, maltodextrin, or mixtures thereof.

Suitable lubricants, for use in a tablet, may be present in amounts from about 0.1% to about 5% by weight, and include, but are not limited to, talc, silicon dioxide, stearic acid, calcium, zinc or magnesium stearate, sodium stearyl fumarate and the like.

Suitable binders, for use in a tablet, include, but are not limited to, gelatin, polyethylene glycol, sugars, gums, starch, polyvinyl pyrrolidone, hydroxypropyl cellulose, hydroxypropylmethyl cellulose and the like. Suitable diluents, for use in a tablet, include, but are not limited to, mannitol, xylitol, lactose, dextrose, sucrose, sorbitol, microcrystalline cellulose and starch.

Suitable solubilizers, for use in a tablet, may be present in amounts from about 0.1% to about 3% by weight, and include, but are not limited to, polysorbates, sodium lauryl sulfate, sodium dodecyl sulfate, propylene carbonate, diethyleneglycol monoethyl ether, dimethyl isosorbide, polyethylene glycol (natural or hydrogenated) castor oil, HCOR™ (Nikkol), oleyl ester, Gelucire™, caprylic/caprylic acid mono/diglyceride, sorbitan fatty acid esters, and Solutol HS™.

Parenteral Administration

Compounds of the present disclosure may be administered directly into the blood stream, muscle, or internal organs. Suitable means for parenteral administration include intravenous, intra-muscular, subcutaneous intra-arterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are aqueous solutions containing excipients, including salts, buffering agents and isotonic agents.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Topical Administration

Compounds of the present disclosure may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can also be performed by electroporation, iontophoresis, phonophoresis and the like. Compositions for topical administration may be formulated as immediate or modified release, including delayed or sustained release.

Manufacturing Method of the Compounds According to the Present Disclosure

The present disclosure further provides a manufacturing method of the compound of Chemical Formula 1 or a pharmaceutically acceptable salt thereof, wherein the method comprises (S1) coupling a compound of Chemical Formula 3 and Chemical Formula 4, (S2) deprotecting the protecting group A, and then (S3) deprotecting the protecting group B.

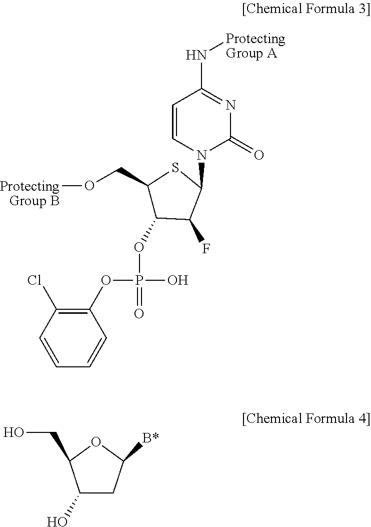

in Chemical Formulae 3 and 4, B* is NH₂-protected adenine, NH₂-protected guanine, NH₂-protected cytosine or thymine.

The manufacturing method can prevent the production of undesired regioisomers.

In another embodiment, there is provided a manufacturing method wherein the protecting group B is monomethoxytrityl. The deprotection of the protecting group B is performed under acidic conditions. Preferably, the acidic conditions is made by acetic acid.

In yet another embodiment, there is provided a manufacturing method wherein the protecting group A is benzoyl. Preferably, the deprotection of the protecting group A is performed by NH₄OH.

In the manufacturing method of the present disclosure, the NH₂ group of the NH₂-protected adenine, NH₂-protected guanine, and NH₂-protected cytosine is protected by benzoyl or isobutyryl.

### Advantageous Effects of Invention

The present disclosure provides a compound having better anti-cancer activity and/or (physicochemical or pharmacokinetic) property than other nucleoside analogs, a pharmaceutical composition having the compound as an effective agent, a medical use, particularly for treating cancers, of the compound, and a method of treatment comprising administering the compound to a subject in need of such treatment or prevention.

### Mode for the Invention

Hereinafter, the present disclosure is described in considerable detail with examples to help those skilled in the art understand the present disclosure. However, the following examples are offered by way of illustration and are not intended to limit the scope of the invention. It is apparent that various changes may be made without departing from the spirit and scope of the invention or sacrificing all of its material advantages.

Preparation of Compounds of the Present Disclosure

Reagents and solvents used below were purchased from Aldrich Chemical Co. (Milwaukee, Wis., USA). ¹H-NMR spectra were evaluated with Bruker Avance 300 MHz, Bruker Avance III HD 300 MHz, Bruker Avance 500 MHz NMR spec-trometer and so on.

Below, the illustrating synthetic examples of some compounds of the present disclosure are described, and other compounds can be prepared by the similar method to one described below with different starting or reacting materials.

[Preparation of Compounds of Chemical Formula 1]

The below 4 compounds (ME20180191-1 to -4) were prepared as follows:

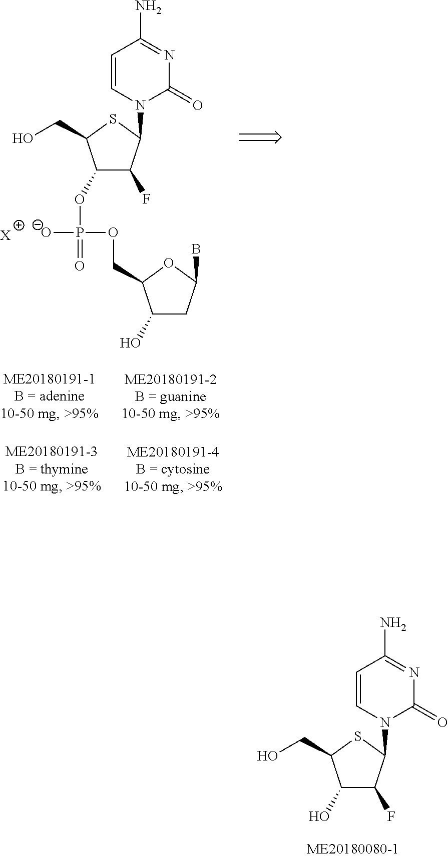

The syntheses of the compounds were started with the preparation of target ME20180191-4. First, ME20180080-1 was TES-protected, which gave compound 1 in quantitative yield.

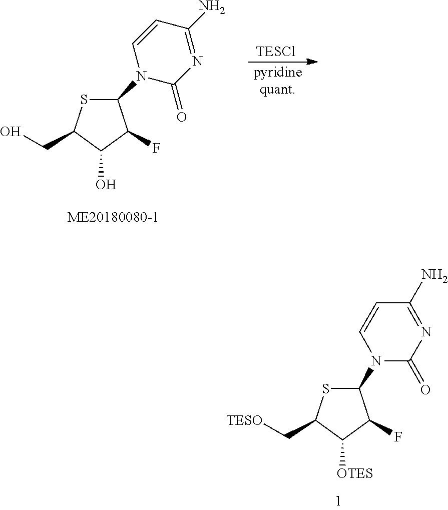

The 4 target compounds were prepared via key intermediate 20.

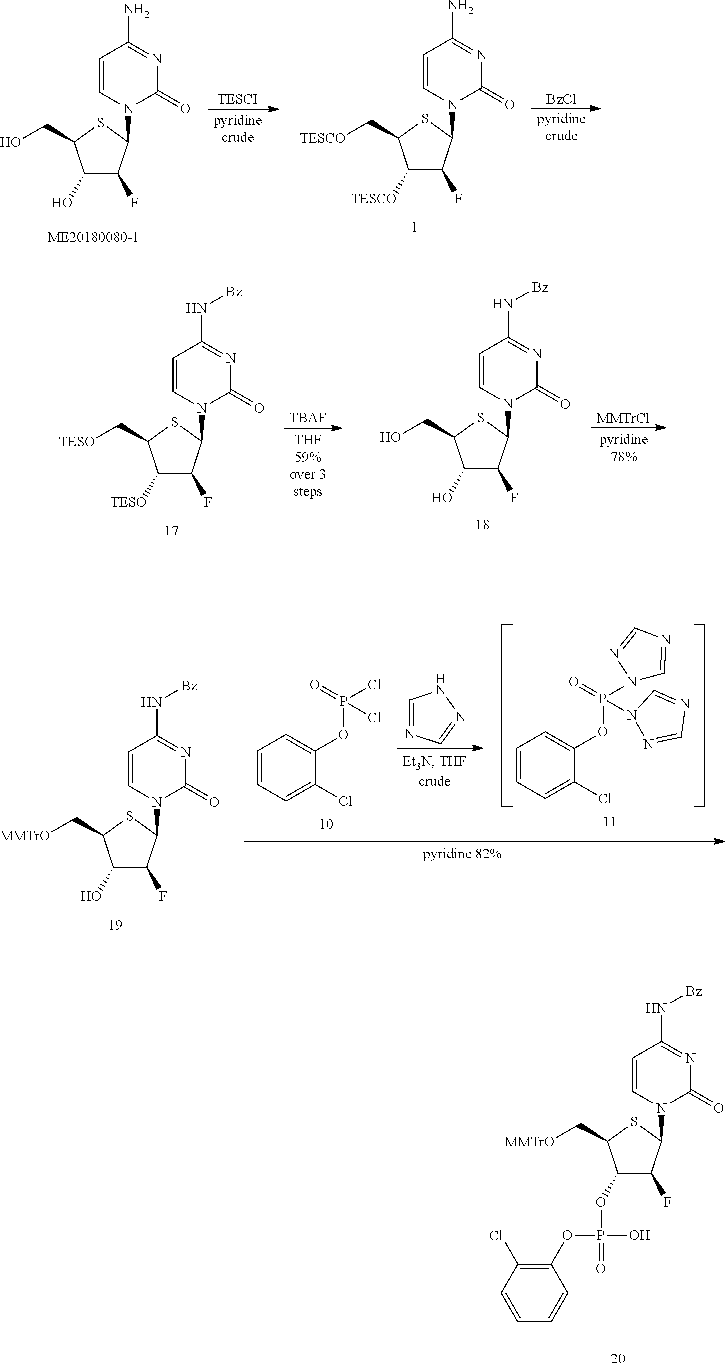

This sequence was started with the TES-protection of ME20180080-1 yielding compound 1, which was benzoylated leading to 17. Subsequent, TES-deprotection afforded compound 18 in a yield of 59% over 3 steps. Next, selective MMTr-protection of the primary alcohol gave 19 in 78% yield. Phosphorylation of 19 was performed using in situ prepared reagent 11. After purification of the obtained product by silica gel flash column chromatography, key intermediate 20 was obtained in 78% yield.

Phosphate 20 was then coupled to thymidine (14), mediated by TPSNI. After purification by flash column chromatography, phosphotriester 21 was obtained in 52%. MMTr-deprotection was performed using dichloroacetic acid (DCA) to afford 22 in 78% yield. Deprotection of the remaining protecting groups was performed using aqueous ammonia. Unexpectedly, the analysis of the crude reaction product by LC-MS showed two equally high peaks with very similar retention times, both with the correct mass of the desired compound. Also, 1H-NMR analysis of the obtained material showed two sets of signals.

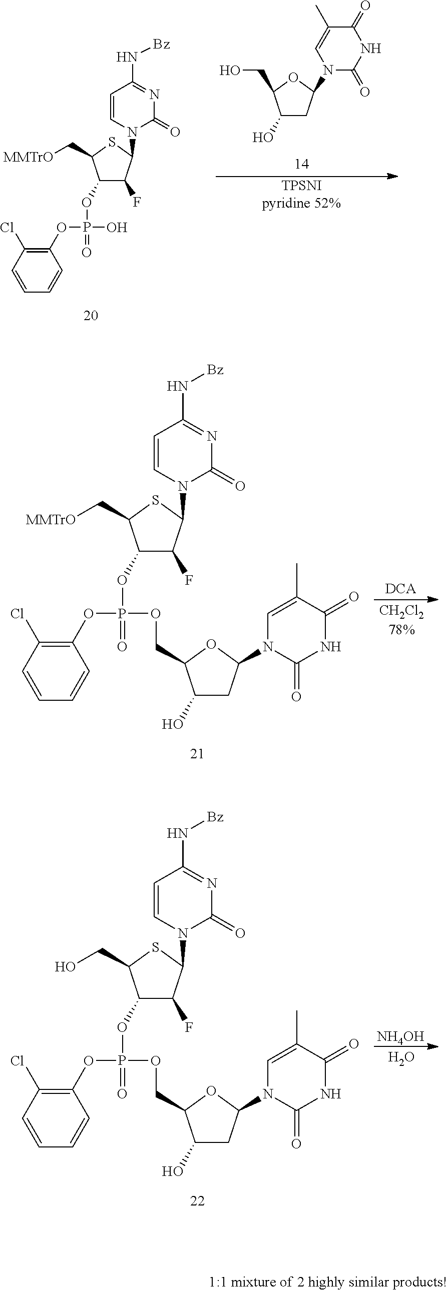

It could be excluded that a mixture of two diastereomeric salts was obtained, because high-temperature NMR, high-temperature HPLC and treatment of a sample with strong acid gave no change in the ratio of the two products. Meanwhile, the purification of the thymidine-derivative by preparative HPLC had been performed successfully. The 1H-NMR gave clear spectra without doubled signals.

The same behavior was observed with the cytidine-derivative. Phosphate 27 was isolated in 45% yield after purification by flash column chromatography. After removal of the protecting groups, again a 1:1 mixture of two highly similar compounds was obtained.

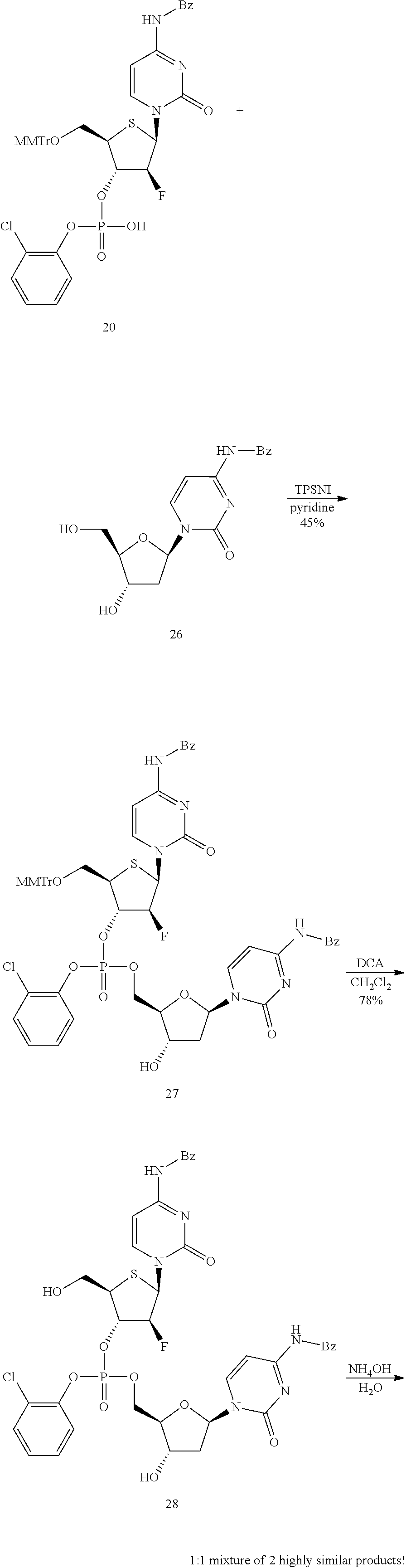

Then, we hypothesized that the mixture of products might consist of two re-gioisomeric products, resulting from unspecific coupling of building block 20 to the 5′- or 3′-prime hydroxyl groups of the nucleosides. The mixture of products would then consist of the desired 3′-5′-coupled dinucleotide and the undesired 3′-3′-coupled dinucleotide.

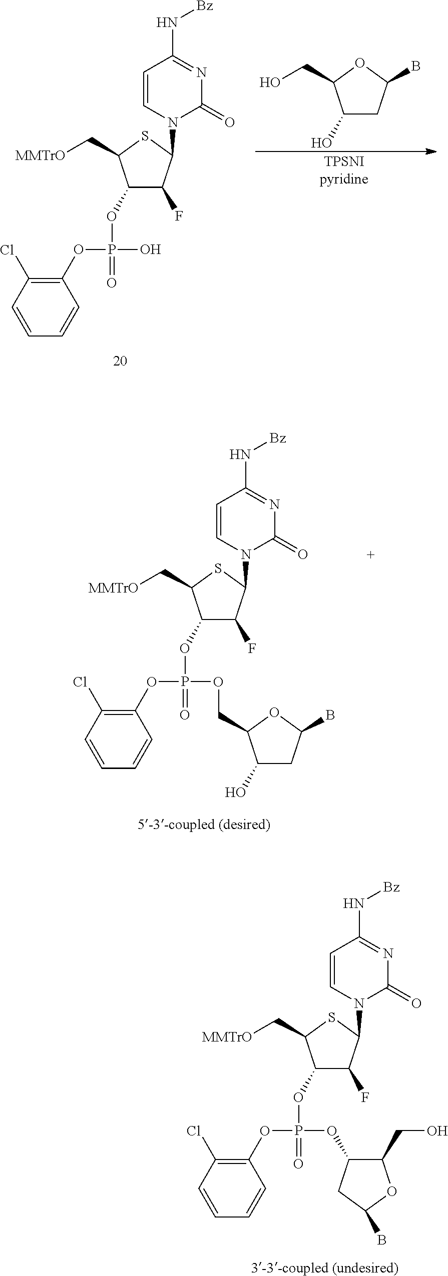

To study this hypothesis, 5′-protected cytidine 26 was used. If indeed the mixture of compounds is caused by the formation of regio-isomers, this should give only the undesired 3′-3′-coupled compound.

Thus, compound, 20 was coupled with 5′-DMTr-26, affording 32.

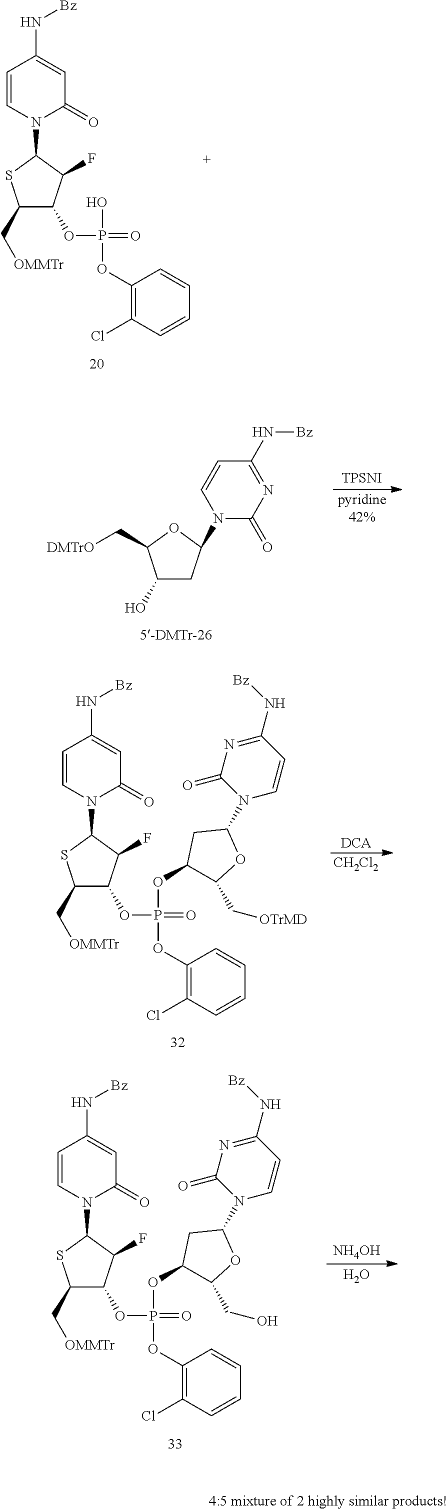

Surprisingly, after removal of the protecting groups, again a mixture of two highly similar products was obtained, both with the mass of the expected product. However, these products were not identical to the cytidine-products we had obtained before. Therefore, we had to conclude that the mixture of products that we obtained was not caused by unspecific coupling of the nucleosides.

As it was now clear that the mixture of products was not composed of a mixture of the desired 3′-5′-coupled dinucleotide and the undesired 3′-3′-coupled dinucleotide, we looked into another possibility for the formation of 2 regiomeric compounds. It might be possible that during the basic deprotection step, an intramolecular attack of 5′-hydroxyl of the ME20180080-1 moiety onto the phosphotriester occurs to form a cyclic intermediate. If this intermediate would subsequently undergo aspecific hydrolysis, two regioisomeric products would be formed.

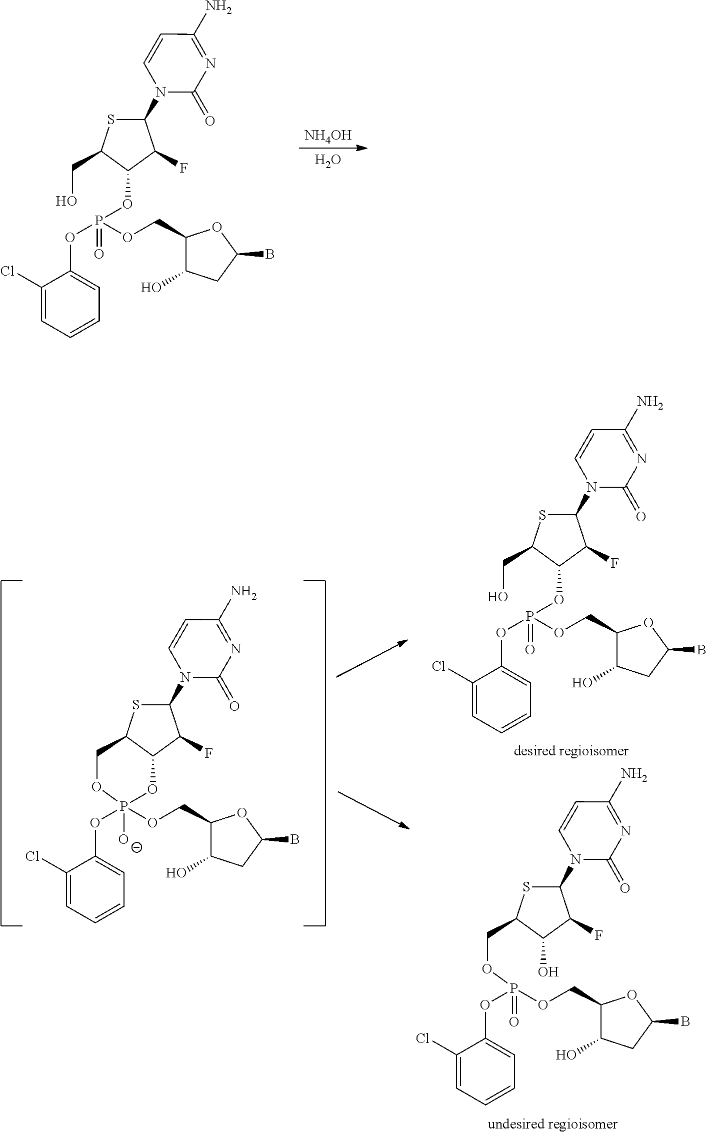

In order to investigate this, we aimed to perform the final deprotection steps in the reversed order, keeping the 5′-hydroxyl group of ME20180080-1 protected during the basic hydrolysis.

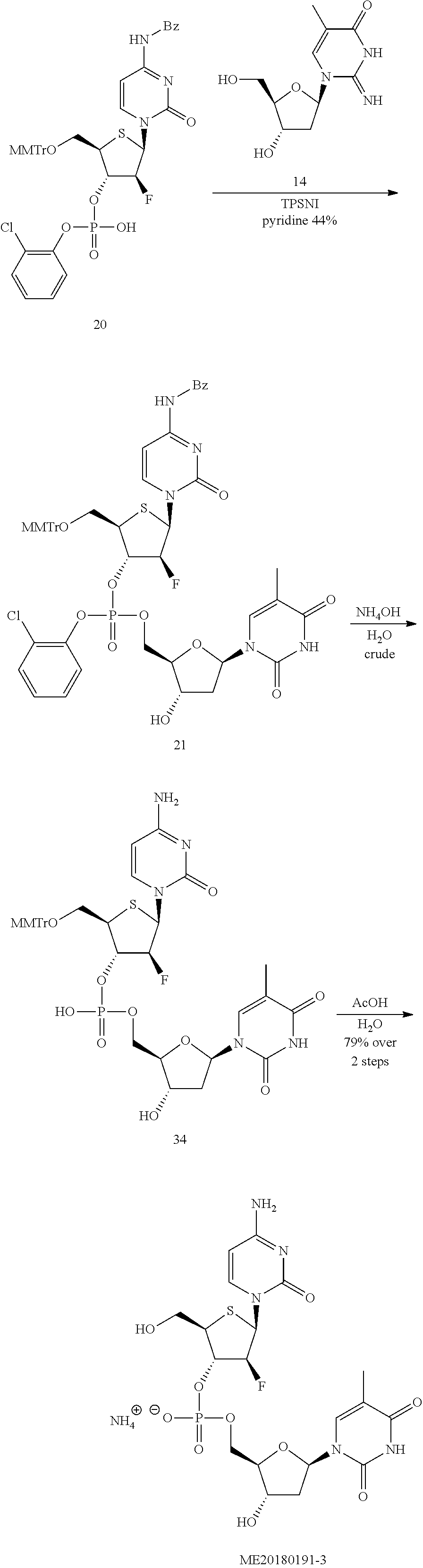

Ammonia mediated deprotection of compound 21 gave complete conversion to 34. This was used crude for the removal of MMTr under acidic conditions. Gratifyingly, a single compound was formed, which was purified by preparative HPLC. This was shown to be the desired compound ME20180191-3, which was obtained in sufficient amount and purity.

Knowing now that the formation of the mixture of products can be prevented by reversing the order of the final deprotection steps, the synthesis of the remaining 3 target compounds was performed. TPSNI-mediated coupling of intermediate 20 with the A, C, and G-nucleosides, afforded phosphotriesters 30, 24 and 27, respectively.

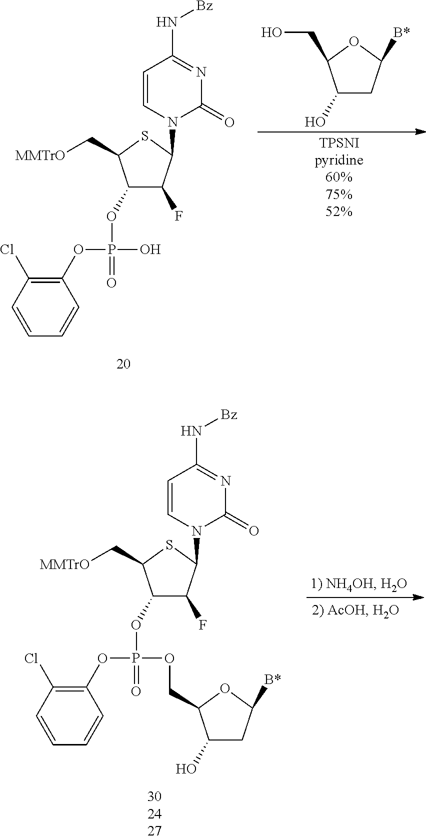

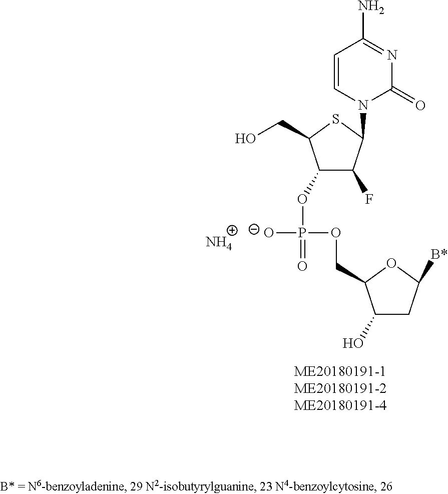

Subsequently, removal of the benzoyl and chloro-phenyl moieties were performed under basic conditions, followed by acidic MMtr removal, affording the crude target molecules as single compounds. Purification of these compounds by preparative HPLC was performed.

[Preparation of Compounds of Chemical Formula 2]

The below 4 compounds (ME20190021-1 to -4) were prepared as follows:

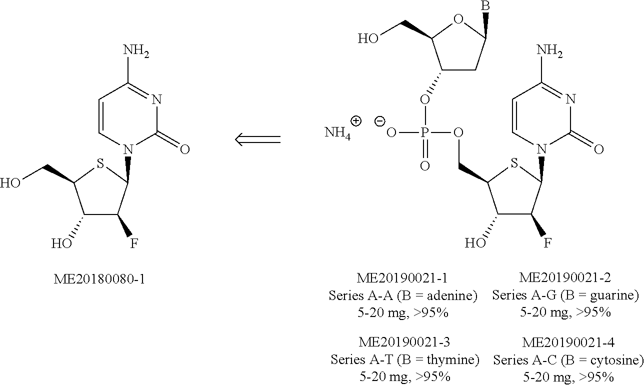

The synthesis was started with mono-silylated building block 1, prepared in ME20190020. This compound was treated with MMTrC1 to obtain bis-tritylated derivative 2. Deprotection of the TBMDS group gave compound 3 in 68% yield.

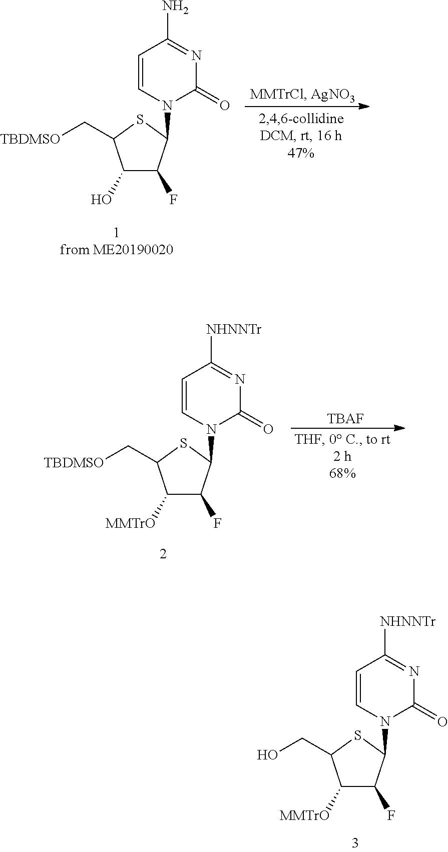

The required phosphates 9A/G/T/C were synthesized.

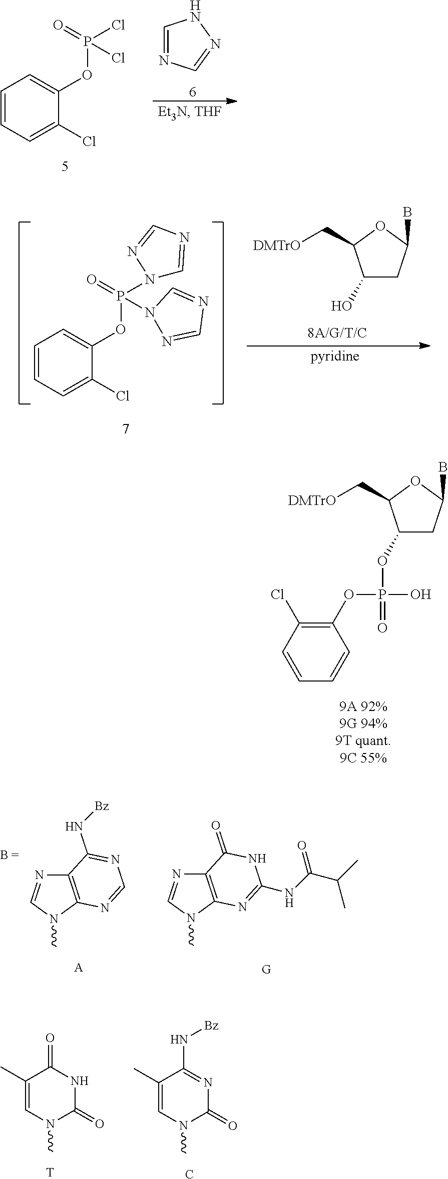

The preparation of the targets ME20190021-1-4 was started with the MSNT mediated couplings of phosphates 9A/G/T/C with alcohol 3 to compounds 10A/G/T/C. Removal of the base-labile protecting groups was performed in aqueous ammonia. After purification by preparative MPLC, compounds 11A/G/T/C were obtained. Acidic deprotection of the trityl groups was performed using aqueous acetic acid, leading to the final compounds. Purification by preparative HPLC afforded sufficient amounts of the target compounds in good purities.

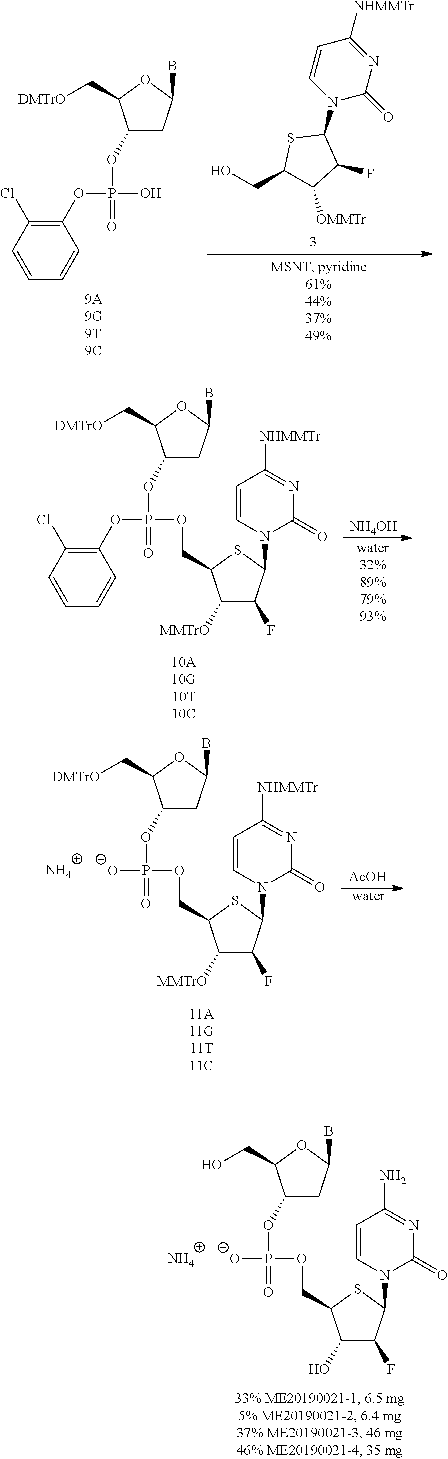

Results of ¹H NMR tests of the above compounds are written in Table 1 below.

TABLE 1

| Compound | |
|---|---|
| No. | ¹H NMR |
| ME20180080-1 | ¹H NMR (400 MHz, DMSO- d₆) δ 7.99 (d, J = 7.4 Hz, 1H), 7.27 (bs, 1H), |
| | 7.20 (bs, 1H), 6.46 (dd, J = 14.7, 5.2 Hz, 1H), 5.86 (d, J = 4.8 Hz, 1H), |
| | 5.77 (d, J = 7.4 Hz, 1H), 5.24 (t, J = 5.4 Hz, 1H), 4.92 (dt, J = 51.0, 5.3 |
| | Hz, 1H), 4.25 (dq, J = 10.3, 4.9 Hz, 1H), 3.72 (dt, J = 11.2, 5.7 Hz, 1H), |
| | 3.60 (dt, J = 11.6, 6.1 Hz, 1H), 3.26-3.18 (m, 1H). |
| ME20180191-1 | ¹H NMR (400 MHz, Deuterium Oxide) δ 8.33 (s, 1H), 8.13 (s, 1H), 7.93 |
| | (dd, J = 7.6, 1.7 Hz, 1H), 6.38 (t, J = 6.7 Hz, 1H), 6.32 (dd, J = 17.3, 4.7 |
| | Hz, 1H), 5.88 (d, J = 7.7 Hz, 1H), 4.92 (dt, J = 49.9, 4.6 Hz, 1H), 4.71 (dt, |
| | J = 6.6, 4.0 Hz, 1H), 4.51 (tt, J = 8.9, 4.1 Hz, 1H), 4.21-4.16 (m, 1H), |
| | 4.07-3.98 (m, 2H), 3.76 (dd, J = 12.1, 5.9 Hz, 1H), 3.61 (ddd, J = 11.9, |
| | 7.2, 1.8 Hz, 1H), 3.46-3.40 (m, 1H), 2.81 (dt, J = 13.6, 6.7 Hz, 1H), 2.55 |
| | (ddd, J = 13.9, 6.5, 4.2 Hz, 1H). |
| ME20180191-2 | ¹H NMR (400 MHz, Deuterium Oxide) δ 8.09 (dd, J = 7.8, 1.7 Hz, 1H), |
| | 8.03 (bs, 1H), 6.43 (dd, J = 17.8, 4.6 Hz, 1H), 6.25 (t, J = 6.7 Hz, 1H), |
| | 6.02 (bs, 1H), 5.04 (dt, J = 49.8, 4.5 Hz, 1H), 4.76-4.68 (m, 3H), 4.61 (tt, |
| | J = 8.9, 4.0 Hz, 1H), 4.22-4.15 (m, 1H), 4.14-4.02 (m, 2H), 3.83 (ddd, J = |
| | 11.7, 6.0, 1.4 Hz, 1H), 3.68 (ddd, J = 11.7, 7.2, 1.9 Hz, 1H), 3.58-3.49 (m, |
| | 1H), 2.84 (dt, J = 13.7, 6.7 Hz, 1H), 2.52 (ddd, J = 14.0, 6.7, 4.2 Hz, 1H). |
| ME20180191-3 | ¹H NMR (400 MHz, Deuterium Oxide) δ 8.44 (dd, J = 7.9, 1.3 Hz, 1H), |
| | 7.67 (d, J = 1.2 Hz, 1H), 6.42 (dd, J = 13.2, 5.1 Hz, 1H), 6.30 (t, J = 6.8 |
| | Hz, 1H), 6.19 (d, J = 7.9 Hz, 1H), 5.29 (dt, J = 49.7, 5.3 Hz, 1H), |
| | 4.60-4.52 (m, 1H), 4.18-4.03 (m, 3H), 3.90 (ddd, J = 12.0, 5.5, 1.7 Hz, |
| | 1H), 3.84 (ddd, J = 12.0, 6.3, 1.5 Hz, 1H), 3.66-3.60 (m, 1H), 2.42-2.29 |
| | (m, 2H), 1.88 (d, J = 1.2 Hz, 3H). |
| ME20180191-4 | ¹H NMR (400 MHz, Deuterium Oxide) δ 8.29 (dd, J = 7.6, 1.2 Hz, 1H), |
| | 7.89 (d, J = 7.7 Hz, 1H), 6.40 (dd, J = 11.3, 5.4 Hz, 1H), 6.23 (t, J = 6.5 |
| | Hz, 1H), 6.04 (dd, J = 7.7, 4.9 Hz, 2H), 5.25 (dt, J = 50.1, 5.8 Hz, 1H), |
| | 4.75-4.65 (m, 1H), 4.49 (dt, J = 6.8, 3.9 Hz, 1H), 4.11 (dq, J = 11.1, 2.3 |
| | Hz, 2H), 4.05 (dt, J = 11.9, 4.1 Hz, 1H), 3.95-3.81 (m, 2H), 3.57 (q, J = |
| | 5.7 Hz, 1H), 2.39 (ddd, J = 14.1, 6.5, 4.1 Hz, 1H), 2.23 (dt, J = 13.8, 6.6 |
| | Hz, 1H). |
| ME20190021-1 | ¹H NMR (400 MHz, Deuterium Oxide) δ 8.43-8.39 (m, 2H), 8.25 (s, 1H), |
| | 6.50 (t, J = 6.3 Hz, 1H), 6.36 (t, J = 6.3 Hz, 1H), 6.03 (d, J = 7.8 Hz, 1H), |
| | 5.19 (ddd, J = 50.3, 7.7, 5.9 Hz, 1H), 4.97-4.91 (m, 1H), 4.44 (dt, J = |
| | 12.4, 7.3 Hz, 1H), 4.37 (q, J = 3.6 Hz, 1H), 4.29-4.14 (m, 2H), 3.90 (qd, |
| | J = 12.8, 3.7 Hz, 2H), 3.59-3.51 (m, 1H), 2.99 (dt, J = 13.1, 6.3 Hz, 1H), |
| | 2.81 (dt, J = 13.9, 5.3 Hz, 1H). |
| ME20190021-2 | 1H NMR (400 MHz, Deuterium Oxide) δ 8.25 (d, J = 7.7 Hz, 1H), 8.01 |
| | (s, 1H), 6.38 (t, J = 6.2 Hz, 1H), 6.28 (t, J = 6.4 Hz, 1H), 5.87 (d, J = 7.6 |
| | Hz, 1H), 5.16 (ddd, J = 50.5, 7.6, 5.9 Hz, 1H), 4.91-4.84 (m, 1H), 4.42 |
| | (dt, J = 12.3, 7.4 Hz, 1H), 4.29 (q, J = 3.8 Hz, 1H), 4.25-4.13 (m, 2H), |
| | 3.84 (qd, J = 12.6, 4.0 Hz, 2H), 3.55-3.49 (m, 1H), 2.93 (dt, J = 13.4, 6.5 |
| | Hz, 1H), 2.71 (ddd, J = 13.9, 5.9, 4.1 Hz, 1H). |
| ME20190021-3 | ¹H NMR (400 MHz, Deuterium Oxide) δ 8.44 (d, J = 7.7 Hz, 1H), 7.66 |
| | (d, J = 1.5 Hz, 1H), 6.38 (dd, J = 7.4, 5.7 Hz, 1H), 6.27 (t, J = 6.7 Hz, |
| | 1H), 6.13 (d, J = 7.8 Hz, 1H), 5.17 (ddd, J = 50.3, 7.4, 5.8 Hz, 1H), |
| | 4.81-4.75 (m, 1H), 4.43 (dt, J = 12.1, 7.1 Hz, 1H), 4.23-4.09 (m, 3H), |
| | 3.82 (qd, J = 12.6, 4.0 Hz, 2H), 3.54-3.48 (q, J = 5.1 Hz, 1H), 2.56 (ddd, J = |
| | 14.2, 6.2, 3.9 Hz, 1H), 2.43 (dt, J = 13.9, 6.7 Hz, 1H), 1.84 (d, J = 1.2 |
| | Hz, 3H). |
| ME20190021-4 | ¹H NMR (400 MHz, Deuterium Oxide) δ 8.41 (d, J = 7.7 Hz, 1H), 7.93 |
| | (d, J = 7.6 Hz, 1H), 6.37 (t, J = 6.3 Hz, 1H), 6.21 (t, J = 6.1 Hz, 1H), 6.11 |
| | (d, J = 7.7 Hz, 1H), 6.01 (d, J = 7.6 Hz, 1H), 5.17 (ddd, J = 50.3, 7.5, 5.9 |
| | Hz, 1H), 4.78-4.64 (m, 1H), 4.43 (dt, J = 12.2, 7.3 Hz, 1H), 4.25-4.10 (m, |
| | 3H), 3.89 (dd, J = 12.7, 3.2 Hz, 1H), 3.81 (dd, J = 12.8, 4.5 Hz, 1H), |
| | 3.55-3.47 (m, 1H), 2.63 (dt, J = 14.1, 5.6 Hz, 1H), 2.45 (dt, J = 13.6, 6.2 |
| | Hz, 1H). |

Evaluation of Compounds

Anti-cancer property of the compounds according to the present invention was performed as follows:

1. Test sample and compound addition

The test compounds and bortezomib (positive control) were solved in DMSO as 5 mM solution, which was aliquoted, frozen at −20° C. and thawed just prior addition by nanodrop dispensing. Compound treatment of cells started one day after seeding with a final DMSO concentration of 0.1% and was generally performed by nanodrop-dispensing using a Tecan Dispenser. 0.1% DMSO (solvent) and Staurosporine (1.0E-05 M) served as high control (100% viability) and low control (0% viability), respectively.

2. Cell Viability Assay

Cells were cultured in different media. For the assays, cells were seeded in white cell culture-treated flat and clear bottom multiwell plates and incubated at 37° C. before the compound was added. After incubation for 72 h at 37° C. at 5% or 10% CO₂ dependent on the medium, cell plates were equilibrated to room temperature for one hour, CellTiterGlo reagent (Promega) was added and luminescence was measured ap-proximately an hour later using a luminometer.

3. Evaluation of Raw Data

Raw data were converted into percent cell viability relative to the high and low control, which were set to 100% and 0%, respectively. IC₅₀ calculation was performed using GraphPad Prism software with a variable slope sigmoidal response fitting model using 0% viability as bottom constraint and 100% viability as top constraint. As compounds repeatedly showed only partial inhibition, IC₅₀ values were also determined without bottom constraints.

The results are shown in Table 2 below.

TABLE 2

| | | ME20180191-2 | ME20180191-2 | ME20180191-1 | ME20180191-3 | ME20180191-4 | Bortezomib |
|---|---|---|---|---|---|---|---|
| Cell | | IC ₅₀ | Residual | IC ₅₀ | IC ₅₀ | IC ₅₀ | IC ₅₀ |
| line | Origin | (nM) | Cell % | (nM) | (nM) | (nM) | (nM) |
| A172 | Brain | 40 | <5 | | | | |
| A375 | Skin | 17 | <5 | 38 | 42 | 41 | 8.5 |
| A549 | NSCLC | 22 | ~5 | | | | |
| Caki-1 | Kidney | 7.7 | <5 | | | | |
| COV434 | Ovary | 51 | ~5 | | | | |
| DU-145 | Prostate | 10 | <5 | 23 | 22 | 22 | 11 |
| H460 | NSCLC | 23 | <5 | 42 | 39 | 43 | 16 |
| HCC38 | Breast | 8.3 | <5 | | | | |
| HCT116 | Colorectal | 9.9 | ~5 | | | | |
| HL-60 | AML | 5.2 | <5 | | | | |
| HT-1080 | Sarcoma | 27 | ~5 | | | | |
| Hutu 80 | Stomach | 4.3 | <5 | | | | |
| KG-1 | AML | 21 | <5 | | | | |
| LN229 | Brain | 86 | <5 | | | | |
| M07e | AML | 16 | <5 | | | | |
| Molm13 | AML | 10 | <5 | | | | |
| MV4-11 | AML | 2.2 | <5 | 7 | 6.4 | 6.4 | 3.8 |
| NCI-H1048 | NSCLC | 6.9 | <5 | | | | |
| NCI-H2110 | NSCLC | 12 | <5 | | | | |
| NCI-H2286 | NSCLC | 7.4 | <5 | 16 | 16 | 18 | 6.6 |
| NCI-H292 | NSCLC | 10 | <5 | | | | |
| OCI-AML5 | AML | 30 | <5 | | | | |
| SK-ES-1 | Sarcoma | 14 | <5 | | | | |
| SK-N-MC | Brain | 5.6 | <5 | | | | |
| SNU-1 | Stomach | 7.6 | <5 | | | | |
| U118MG | Brain | 20 | <5 | | | | |
| U937 | AML | 12 | <5 | | | | |

As shown in the above Table 2, the compounds of the present disclosure were useful for inhibiting various cancer cell lines. Particularly, the ME2018191-2 was more effective than other test compounds, and was effective for a great variety of cancer cell lines including brain tumor (A172, LN229, SK-N-MC, U118MG), non-small cell lung cancer (A549, H460, NCI-H1048, NCI-H2110, NCI-H2286, NCI-H292), acute myelogenous leukemia (HL-60, KG-1, M07e, Molm13, MV4-11, OCI-AML5, U937), stomach cancer (SNU-1, Hutu 80), kidney cancer (Caki-1), colon cancer (HCT-116), prostate cancer (DU-145), ovarian cancer (COV434), skin cancer (A375), and sarcoma (SK-ES-1, HT-1080). Particularly, the dinucleotide compound of the present disclosure showed 1-100 nM of IC₅₀ and less than 5% of residual cells for most cancer cell lines, which means that the compounds of the present disclosure have very outstanding anti-cancer effect.

All mentioned documents are incorporated by reference as if herein written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

1. A compound of Chemical Formula 1 or 2: 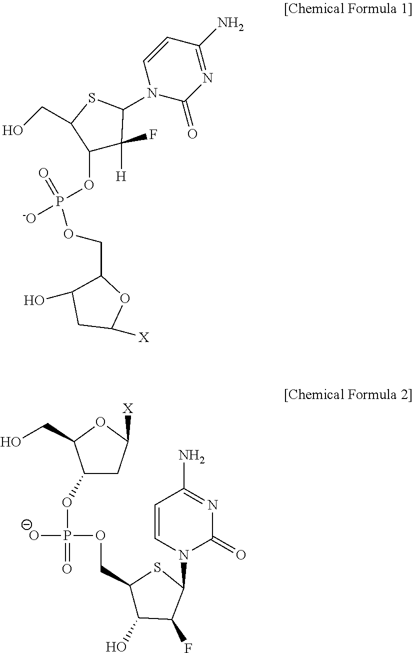
or a pharmaceutically acceptable salt thereof,
in Chemical Formulae 1 and 2, X is adenine, guanine, cytosine or thymine.

2. The compound of claim 1, wherein the compound has a structure of Chemical Formula 1 and X is guanine.

3. A composition comprising a compound of claim 1, or a pharmaceutically acceptable salt thereof, and a pharmaceutically acceptable carrier.

4.-6. (canceled)

7. A method for treating or preventing cancer comprising administering to a subject in need thereof a therapeutically effective amount of a compound of claim 1, or pharmaceutically acceptable salt thereof.

8. The method of claim 7, wherein the cancer is brain tumor, non-small cell lung cancer, acute myelogenous leukemia, stomach cancer, kidney cancer, colon cancer, prostate cancer, ovarian cancer, skin cancer, or sarcoma.

9. The method of claim 8, wherein the cancer is skin cancer, prostate cancer, non-small cell lung cancer, or acute myelogenous leukemia.

10. A method of manufacturing a compound of Chemical Formula 1 of claim 1 or a pharmaceutically acceptable salt thereof, wherein the method comprises (S1) coupling a compound of Chemical Formula 3 and Chemical Formula 4, (S2) deprotecting the protecting group A, and then (S3) deprotecting the protecting group B. 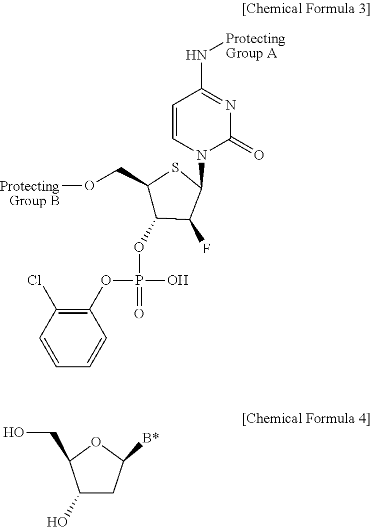
in Chemical Formulae 3 and 4, B* is NH₂-protected adenine, NH₂-protected guanine, NH₂-protected cytosine or thymine.

11. The method of claim 10, wherein the protecting group B is monomethoxytrityl.

12. The method of claim 10, wherein the deprotecting of (S3) is performed under acidic conditions.

13. The method of claim 12, wherein the acidic conditions is made by acetic acid.

14. The method of claim 10, wherein the protecting group A is benzoyl.

15. The method of claim 10, wherein the deprotecting of (S2) is performed by NH4OH.

16. The method of claim 10, wherein the NH₂ group of the NH₂-protected adenine, NH₂-protected guanine, and NH₂-protected cytosine is protected by benzoyl or isobutyryl.
